(19) United States
(12) Patent Application Publication
(10) Pub. No.: US 2004/0058422 A1
(43) Pub. Date: Mar. 25, 2004

(54) Process for preparing (-)- menthol and similar compounds

(76) Inventors: Jennifer Ann Chaplin, San Diego, CA (US); Neil Stockenstrom Gardiner, Pretoria (ZA); Robin Kumar Mitra, Benoni (ZA); Christopher John Parkinson, Modderfontein (ZA); Madrie Portwig, Greenside (ZA); Butana Andrew Mboniswa, Edenvale (ZA); Melanie Daryle Evans-Dickson, Livingstone (ZA); Dean Brady, Midrand (ZA); Stephanus Francois Marais, Garsfontein (ZA); Shavani Reddy, Edenvale (ZA)

Correspondence Address: STERNE, KESSLER, GOLDSTEIN & FOX PLLC, 1100 NEW YORK AVENUE, N.W., WASHINGTON, DC, 20005, US

(21) Appl. No.: 10/415,567

(22) PCT Filed: Nov. 1, 2001
(86) PCT No.: PCT/IB01/02047

(30) Foreign Application Priority Data
Nov. 2, 2000 (ZA) 2000/6260

(51) Int. Cl. C12P 7/02

(52) U.S. Cl. 435/155

(57) ABSTRACT

A process of separating a desired (−) stereoisomer which is selected from (−) menthol or an equivalent (−) compound where the isopropyl group is replaced with an isopropanol or an isopropylene group, from a starting material comprising: 40 to 100 m/m % of a mixture of (−)-menthol and (+)-menthol; up to 30 m/m % of a mixture of (−)-isomenthol and (+)-isomenthol; up to 20 m/m % of a mixture of (−)-neomenthol and (+)-neomenthol; and up to 10 m/m % of a mixture of (−)-neoisomenthol and (+)-neoisomenthol or an equivalent (+) mixture where the isopropyl group is replaced with an isopropanol or an isopropylene group, includes the steps of: contacting the starting material with an esterifying agent and a stereospecific enzyme which is a Pseudomonas lipase enzyme which stereoselectively esterifies the —OH group of the desired (−) steroisomer, for a time sufficient to convert a desired percentage of the desired (−) stereoisomer to a desired (−) esterified compound where the —OH group is converted to a group —O—C(O)—R4, wherein R4 is an alkyl or an aryl group, to give a first reaction product including the desired (−) esterified compound, the organic solvent, the unconverted stereoisomers, excess esterifying agent and by-products of the reaction; and separating the desired (−) esterified compound from the first reaction product. The process is of particular application for the production of (−)-menthol.

### BACKGROUND TO THE INVENTION

THIS invention relates to a process for producing (−)-menthol and similar compounds.

Menthol has been the subject of much research in the flavour industry. The molecule of menthol has three asymmetric carbon atoms, and hence, a total of eight optically active isomers are possible. The eight isomers are (−)-menthol, (+)-menthol, (−)-isomenthol, (+)-isomenthol, (−)-neomenthol, (+)-neomenthol, (−)-neoisomenthol and (+)-neoisomenthol. Of all of these isomers only (−)-menthol has a strong refreshing character and is widely used in perfumes and medicines. Thus, the isolation of (−)-menthol from the other isomers is industrially important.

As previously discussed, reacemic menthol contains four stereoisomeric pairs of menthols. The isolation of (−)-menthol from this isomeric mixture can be performed chemically via crystallisation, freeze-drying or distillation.

The hydrogenation of thymol results in the formation of eight menthol isomers which can then be esterified and the (−)-menthyl ester selectively hydrolysed by microbial enzymes to yield (−)-menthol. The use of enzymes, either as free enzymes or part of a whole cell system, has been widely studied for the resolution of (−)-menthol from racemic mixtures.

A large variety of bacteria, fungi and yeasts have been identified with the ability to perform the hydrolysis of menthyl esters. It was reported in Biotechnol. Gen. Engineer. Rev. 6:271-320 by Y Mikami (1988) that, of the microorganisms capable of hydrolysing menthyl acetates, bacteria and fungi also hydrolyse isomenthyl acetate.

In a patent issued to Takasago Perfumery Co Ltd. (U.S. Pat. No. 3,607,651; Sep. 21, 1971,) the following organisms were claimed to possess a carboxylic hydrolase: Penicillium, Gliocladium, Trichoderma, Geotrichum, Aspergillus, Pullularia, Fusrium, Absida, Cunninghamella, Rhizopus, Actinomucor, Chlamydomucor, Mucor, Gibberella, Streptomyces, and Bacillus. They were all shown to hydrolyse (−)-menthyl esters as well as (−)-isomenthyl esters. The two isomers of menthol formed can then be separated using rectification, recrystallisation and chromatography.

Some of the yields obtained in the examples are: 47.5% conversion of (±)-menthyl acetate with Absidia hyalospora in 24 hours; 28.8% (−)-menthol and 17.4% (−)-isomenthol was also obtained from a mixture of menthyl acetates (5.8% (±)-neomenthol; 30.4% (±) isomenthol; 63.8% (±)-menthol and others) in 24 hours with Trichoderma viride; 50.3% from (±)menthyl acetate with Bacillus subtilus var niger after 48 hours.

A novel process for the stereoselective hydrolysis of the monochloroacetate of (±)-menthol has been developed using Pseudomonas sp. NOF-5, which appears to have been reclassified as Alginomonas nonfermentans NOF-5.

The organism was shown to hydrolyse (±)-isomenthyl acetate to (−)-isomenthol and also to hydrolyse (±)-neoisomenthyl acetate nonstereospecifically. (See again U.S. Pat. No. 3,607,651).

The most widely studies lipase is that of Candida cylindracea (which has been reclassified as Candida rugosa). It was studied for the separation of (−)-menthol from the esters formed by the esterification of a menthol mixture obtained from the Haarmann-Reimer process, which consisted of 55% (±)-menthol, 29% (±)-neomenthol, 14% (±)-isomenthol and 2% (±)-neoisomenthol. The order of selectivity for the menthol isomers by the C. rugosa lipase used (lipase MY, Meito Sangyo Ltd) was (−)-menthol (100%), (−)-isomenthol (48%), (−)-neoisomenthol (35%), (−)-neomenthol (3.3%), (+)-menthol (2%), (+)-neoisomenthol (0.6%) and (+)-neomenthol (<0.1%). The (−)-menthol and (−)-isomenthol esters were converted first; this occurred at a conversion ratio of 35%, after which the (−)-neomenthol, (+)-menthol and (+)-isomenthol esters were consumed. It was therefore not possible to isolate (−)-menthol from its other isomers; a two-step reaction was then attempted using lipase-catalysed ester synthesis to obtain a mixture enriched in (−)-menthol ester, followed by lipase catalyst ester solvolysis, resulting in (−)-menthol. The process was not successful in completely eliminating the (−)-isomenthol and the purity was not considered adequate for industrial scale. (See Bioflavour '87, C Triantaphylides et al, (1988) Walter dr Gruter & Co. Berlin 531-541).

None of the microorganisms and enzymes described above have the ability to react with (−)-menthol with a high degree of selectivity when the (−)-menthol is included in a mixture with (+)-menthol and the other stereoisomers, isomenthol, neomenthol and neoisomenthol.

In PCT/IB 01/01008 there is disclosed a process of separating a single desired stereoisomer from a racemic mixture of eight stereoisomers of a compound of the formula III by contacting the racemic mixture in a suitable organic solvent with an esterifying agent and a stereospecific enzyme which stereoselectively esterifies the —OH group of the desired stereoisomer, for a time sufficient to convert a desired percentage of the desired stereoisomer to a compound of the formula IV, to give a first reaction product including the compound of the formula IV, the organic solvent, the unconverted stereoisomers of the compound of the formula III, excess esterifying agent and by-products of the reaction; and then separating the compound of the formula IV from the first reaction product.

This invention is an improvement in or modification of the process described above.

### SUMMARY OF THE INVENTION

According to the invention there is provided a process of separating a desired (−) stereoisomer which is selected from (−)-menthol or an equivalent (−) compound where the isopropyl group is replaced with an isopropanol or an isopropylene group, from a starting material comprising:

(a) 40 to 100 m/m % of a mixture of (−)-menthol and (+)-menthol;

(b) up to 30 m/m % of a mixture of (−)-isomenthol and (+)-isomenthol;

(c) up to 20 m/m % of a mixture of (−)-neomenthol and (+)-neomenthol; and

(d) up to 10 m/m % of a mixture of (−)-neoisomenthol and (+)-neoisomenthol,

or an eqivalent (±) mixture where the isopropyl group is replaced with an isopropanol or an isopropylene group (i.e a (+) stereoisomer and a (−) stereoisomer which are respectively equivalent to (+)-menthol and (−)-menthol, and (+)-isomenthol and (−)-isomenthol, and (+)-neomenthol and (−)-neomenthol, and (+)-neoisomenthol and (−)-neoisomenthol except for replacement of the isopropyl group),

including the steps of:

(1) contacting the starting material with an esterifying agent and a stereospecific enzyme which is a Pseudomonas lipase enzyme which stereoselectively esterifies the —OH group of the desired (−) steroisomer, for a time sufficient to convert a desired percentage of the desired (−) stereoisomer to a desired (−) esterified compound where the —OH group is converted to a group, —O—C(O)—R₄, wherein R₄ is an alkyl or an aryl group or hydrogen, to give a first reaction product including the desired (−) esterified compound, the organic solvent, the unconverted stereoisomers, (i.e the (+) stereoisomer of menthol, and the (+) and (−) stereoisomers of isomenthol, neomenthol and neoisomenthol, or its equivalents where the isopropyl group is replaced with an isopropanol or an isopropylene group), excess esterifying agent and by-products of the reaction; and

(2) separating the desired (−) esterified compound from the first reaction product.

Step (2) preferably comprises the sub-steps of:

(2)(a) separating the first reaction product from the enzyme;

(2)(b) removing the organic solvent, the excess esterifying agent, and the by-products of the reaction to give a second reaction product; and

(2)(c) separating the desired (−) esterified compound from the second reaction product to give a third reaction product containing the unconverted stereoisomers.

The process of the invention preferably includes the following step, prior to step (1) of:

(I) subjecting a racemic mixture of the eight stereoisomers of a compound of the formula III

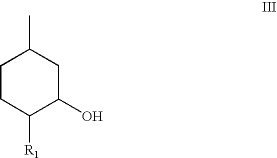

wherein R₁ represents an isopropanol group, an isopropyl group or an isopropylene group,

to a distillation step to separate at least a portion of one or more of the (±) mixtures of isomenthol, neomenthol and neoisomenthol or their equivalents where the isopropyl group is replaced with an isopropanol or an isopropylene group, from the (±) mixture of menthol or its equivalent where the isopropyl group is replaced with an isopropanol or an isopropylene group, to give the starting material for step (1).

The process of the invention preferably includes a further step, step (3) of:

(3) racemizing any unconverted desired (−) stereoisomer, and the other unconverted stereoisomers in the third reaction product, and the six other stereoisomers of the compound of the formula III obtained in step (I) to give a fourth reaction product containing a mixture approaching the thermodynamic equilibrium of the eight stereoisomers and recycling this fourth reaction product to step (I).

The process of the invention preferably includes a further step, step (4) of:

(4) hydrolysing the desired (−) esterified compound to give the desired (−) stereoisomer.

In the process of the invention, where the desired (−)-stereoisomer or the desired (−) esterified compound has an isopropanol group or an isopropylene group, before or after step (4), the desired (−) esterified compound or the desired (−) stereoisomer may be subjected to a reduction step to convert the isopropanol or isopropylene group to an isopropyl group.

The six other stereoisomers of the compound of the formula III may be recycled.

### BRIEF DESCRIPTION OF THE DRAWINGS

FIG. 1 illustrates the structure of various stereoisomers and derivatives of menthol.

### DESCRIPTION OF EMBODIMENTS

The crux of the invention is a process of separating a desired (−) stereoisomer from a starting material containing a specific (±) mixture of stereoisomers, by esterification using a stereospecific enzyme which is a Pseudomonas lipase enzyme.

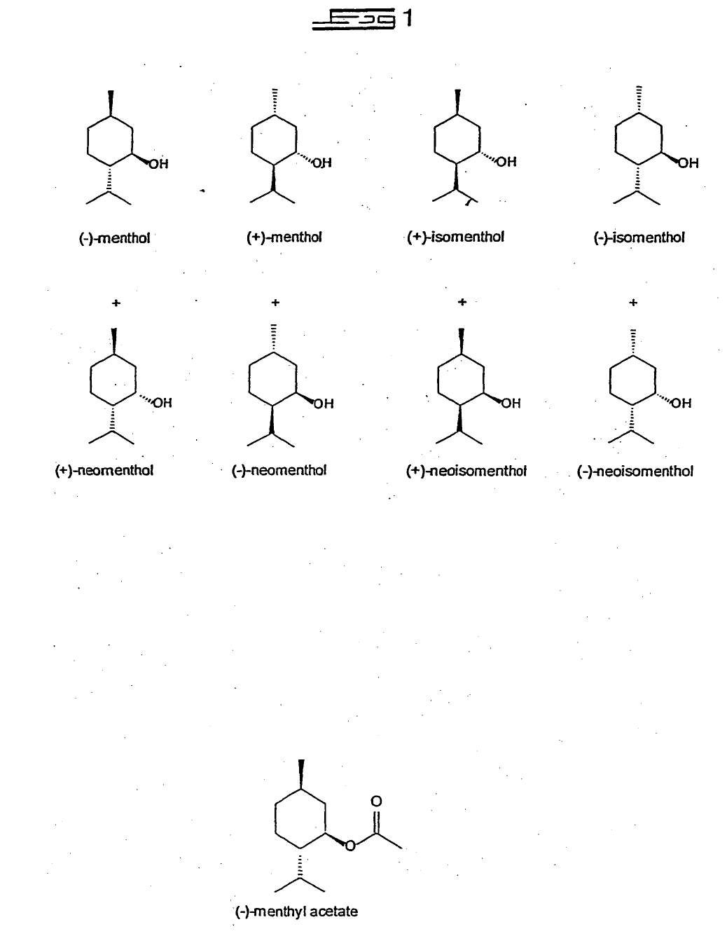

The process is of particular application to the separation of (−)-menthol from a mixture of (±)-menthol and its other six stereoisomers. The structures of (−) menthol and (+)-menthol, as well as the six other stereoisomers of menthol, and the structure of (−)-methyl acetate, are illustrated in FIG. 1.

The stereospecific enzyme is preferably Amano AK lipase enzyme supplied by Amano of Japan.

The enzyme may be used either in the free form or immobilized on a suitable support which may be distomaceous earth.

The conditions used in steps (1) and (2) of the process for the invention are substantially the same as the conditions for steps (1) and (2) of the process disclosed in PCT/IB 01/01008.

For instance, step (1) may be carried out in a suitable organic solvent. The suitable organic solvent may be any solvent typically used for enzyme catalysed esterification reactions including isooctance; n-heptane; decane; methyl cyclohexane; t-butyl methyl ether, xylene; kerosene (C5-C6 paraffins, kerasol 60/115), (C7-C8 paraffins, kerasol 94/125); pentane; cyclohexane; hexane; benzene; butanol; toluene; isopropanol; ethyl lactate; and acetone.

The preferred solvents are t-butyl methyl ether, cyclohexane, hexane, heptane and issoctane, most preferably n-heptane.

The amount of the organic solvent used relative to the starting material is in the range of 0% to 80% of the organic solvent to 100% to 20% of the starting material, preferably in the range of 5% to 80% of the organic solvent to 95% to 20% of the starting material, on a volume basis.

Likewise, the esterifying agent may be any suitable esterifying agent such as for examply vinyl acetate, butyl acetate, octanoic acid, isopropenyl acetate, vinyl butyrate, ethyl lactate and ethyl acetate, with the preferred esterifying agent being vinyl acetate.

The esterifying agent may be used in an molar ratio to the desired (−) stereoisomer of 0,5:1 up to 5:1. The preferred molar ratio of the esterifying agent to the desired (−) stereoisomer is in the range of 1:1 to 2:1 when vinyl acetate is used as the esterifying agent.

The enzyme is preferably used in an amount of from 1 g/l to 60 g/l of the reaction mixture, i.e. the starting material, the suitable organic solvent and the esterifying agent.

The resolution step is preferably carried out at a temperature of from 20° C. to 100° C. inclusive and at atmospheric or higher pressure. When the enzyme is Amano AK, the preferred reaction temperature is about 40° C.

The resolution reaction is continued for a time sufficient to convert a desired percentage of the desired (−) stereoisomer to the desired (−) esterified compound. Generally, it is desirable that as much as possible of the desired (−) stereoisomer is converted to the desired (−) esterified compound without the reaction proceeding to the esterification of the other stereoisomers present in the starting material.

The reaction time is preferably about 24 hours or less when the reaction is performed in batch mode.

The next step, step (2)(a) of the process of the invention is to separate the first reaction product from the enzyme so that the enzyme can be recycled. This may be achieved for example by centrifugation or by filtration.

A major advantage of the process of the invention is that it is possible to recycle the enzyme a number of times to the resolution step, so as to improve the economics of the process.

The necessity for enzyme recycle may be eliminated by use of the enzyme in a continuous system wherein the enzyme is retained within a reactor. The starting material, the organic solvent and the esterifying agent as described above, are fed into the reactor, wherein the desired (−) stereoisomer is esterified to the desired (−) esterified compound to form the first reaction product. The first reaction product typically exits the reactor at a similar rate to the inlet feed, for further processsing. The enzyme may typically be retained within the reactor through the use of membranes, or by immobilisation onto a support material, or through stabilisation by cross-linking.

The next step, step (2)(b) of the process of the invention is to remove the organic solvent, the excess esterifying agent and any by-products, to give a second reaction product including the desired (−) esterified compound and the other unconverted stereoisomers. This may be carried out by distillation in which the organic solvent, e.g the n-heptane and the excess esterifying agent, e.g the vinyl acetate, are taken off at the top of the column as a single stream and recycled back to suitable storage tanks for later re-use.

The next step, step (2)(c) of the process of the invention is to separate the desired (−) esterified compound from the second reaction product leaving a third reaction product containing the unconverted stereoisomers. The separation may be achieved by distillation.

When in the desired (−) esterified compound there is no isopropyl group, the group in question can, through a reduction process, be converted to an isopropyl group, either at this stage, or subsequent to hydrolysis of the ester group as described below. This results in the production of the desired stereoisomer of menthyl ester, or subsequent to hyrolysis, in the production of the desired stereoisomer of menthol.

The next step, step (3) of the process of the invention is to racemise the unconverted stereoisomers in the third reaction product or obtained after step (I), to give a fourth reaction product containing a mixture of all eight stereoisomers and recycling this to the resolution step of the process.

The raceimisation may be achieved over a suitable catalyst with or without hydrogen gas, with or without a solvent, and at atmospheric or greater pressure.

The step may be carried out with or without a solvent. If a solvent is used, the solvent may be any solvent typically used for catalytic hydrogenation, most typically a hydrocarbon or aqueous caustic.

The catalyst used may be any catalyst typically used in homogeneous catalytic hydrogenations such as Pd(OAc)₂ and Ru(PPh₃)₃Cl₂, or in heterogeneous catalytic hydrogenations such as supported palladium, platinum, rhodium, ruthenium, nickel, sponge nickel and 2CuO.Cr₂O₃, or a solid oxide such as celite, CuO, CrO₃, CoO, SiO₂, Al₂O₃, Ba(OH)₂, MnO, Al(iOPr)₃, LnO₂, ZrO and the zeolites. The preferred catalyst is a nickel catalyst.

The reaction may be carried out at any temperature between 80° C. and 300° C. inclusive, preferably at a temperature 180° C. and 220° C.

The hydrogen pressure may be any pressure below 50 bar, preferably between 5 and 35 bar inclusive.

The catalyst loading may be between 0,01 and 20%, preferably between 0,05 and 5%.

At the end of the step, the catalyst is removed or deactivated, and any solvent present is removed.

The next step, step (4) of the process of the invention is to hydrolyse the desired (−) esterified compound to the desired (−) stereoisomer. The reaction may be carried out in the presence of a base which may be a salt of a lower aliphatic alcohol such as sodium methoxide or sodium ethoxide, a metal hydroxide such as KOH, NaOH, or Mg(OH)₂, or amine bases such as NH₄OH.

The reaction may be carried out in any solvent typically used in hydrolysis reactions, such as for example a lower aliphatic alcohol or water. Combinations of the solvents may also be used.

The reaction temperature may be any temperature below the boiling point of the chosen solvent or the reflux temperature of the mixture at the pressure at which the reaction is carried out.

As a final step, the desired (−) stereoisomer may be purified to the desired purity by, for example, distillation or crystallisation.

As indicated above, the starting material must comprise:

(a) 40 to 100 m/m % of a mixture of (−)-menthol and (+)-menthol;

(b) up to 30% m/m % of a mixture of (−)-isomenthol and (+)-isomenthol;

(c) up to 20% m/m % of a mixture of (−)-neomenthol and (+)-neomenthol; and

(d) up to 10 m/m % of a mixture of (−)-neoisomenthol and (+)-neoisomenthol;

or an equivalent (±) mixture where the isopropyl group is replaced with an isopropanol or an isopropylene group.

It is to be noted that in the mixture of (+) and (−) isomers the (+) and (−) isomers need not be present in equal amounts. In other words, for example in the 40 to 100 m/m % of a mixture of (−)-menthol and (+)-menthol, the (−)- and (+)-menthols need not be present in equal amounts.

The preferred composition of the starting material is:

(a) about 80 m/m % of a mixture of (−)-menthol and (+)-menthol,

(b) about 10 m/m % of a mixture of (−)-isomenthol and (+)-isomenthol;

(c) about 6 m/m % of a mixture of (−)-neomenthol and (+)-neomenthol; and

(d) about 4 m/m % of a mixture of (−)-neoisomenthol and (+)-neoisomenthol;

or an equivalent (±) mixture where the isopropyl group is replaced with an isopropanol or an isopropylene group.

The starting material may be obtained, for example, by the process of step (I), i.e a distillation step to separate at least a portion of one or more of the (±) mixtures of isomenthol, neomenthol and neoisomenthol or their equivalents where the isopropyl group is replaced with an isopropanol or an isopropylene group, from the (±) mixture of menthol or its equivalent where the isopropyl groups replaced with an isopropanol or an isopropylene group.

The separation of neomenthol and neoisomenthol from menthol and isomenthol can be achieved by distillation at 150 mbar. The temperatures of the reboiler and reflux streams are typically 150° C. and 120° C. The bottom stream, comprising mainly menthol and isomenthol, can be either fed directly to resolution or further distilled at similar conditions to enrich the menthol component before resolution.

Experimental Work

The results of various experiments carried out in relation to the process of the invention for the separation of (−) menthol from (±) menthol, and the like, are set out below.

### EXAMPLE 1

Lyophilised Amano AK (a Pseudomonas fluorescens lipase enzyme) was obtained from Amano Pharmaceutical Company (Japan). Substrate concentrations of 5, 10, 20 and 40% (m/v) (±)-menthol were added to individual glass reaction vessels. Vinyl acetate was added at a 2:1 molar ratio to (−)-menthol. Heptane was added as a solvent to the final reaction volume of 5 ml. These reaction vessels were incubated in silicon oil baths at 50° C. and stirred on a stirring hot plate. Batch times, unless otherwise stated, were 24 hours. The reaction mixture was then centrifused to separate the products from the enzyme. The supernatant was analysed by GC (% m/m analysis). Of the available (−)-menthol in the above menthol concentrations used, 100%, 86%, 84% and 78% was converted to (−)-menthyl acetate, respectively. The enzyme was recycled by washing with heptane and adding fresh substrate ((±)-menthol, vinyl acetate and heptane) after every recycle.

It can be seen that the use of the enzyme Amano AK provides good conversion of (±)-menthol to the desired (−)-menthol stereoisomer.

### EXAMPLE 2

Reactions (20 ml) were performed at 40° C. in a carousel reaction system containing 10, 25 and 40% synthetic (±)-menthol, vinyl acetate in a ratio of 1:1 with respect to the racemic menthol and heptane. The amounts of enzyme used for the different reactions were: 151 mg Amano AK enzyme per 20 mL for the 10% substrate and 377.5 mg and 604 mg respectively for the 25 and 40% substrate concentrations. Reactions were incubated for 24 hours and then the enzyme separated from the reaction mixture by centrifugation. The supernatant was analysed for % m/m and for (+)-menthol: (−)-mentol ratio using a chiral GC method. Of the available (±) menthol in the above menthol concentrations used, 6, 13 and 15% was converted to (−)-menthyl acetate respectively in the first cycle. Two cycles of the experiment were performed by washing with heptane and adding fresh substrate (±)-menthol, vinyl acetate and heptane) after every cycle. At ˜20% conversion the ee was above 96% in the second cycle.

### EXAMPLE 3

Amano AK enzyme was added to a 2 L baffled, glass jacketed reactor with an overhead impellor. A solution containing 40% m/v (±)-menthol, vinyl acetate at a 2:1 molar ratio to (−)-menthol and heptane was added to a final reaction volume of 1 litre. The reaction mixture was stirred at 500 rpm for 24 hours. Samples from the reactor were centrifuged to separate the products from the enzyme. The supernatant was then submitted for % m/m determination by GC methods as well as chiral analysis. Of the available (−)-menthol, 62% was converted to (−)-menthyl acetate, at an ee of 94%.

### EXAMPLE 4

Batch reactions (200 mL) were performed at 36° C. with 40% m/v (±)-menthol, Amano AK enzyme and vinyl acetate (acyl donor). Methyl tert butyl ether (MTBE) was used as bulk solvent in the reaction. A control reaction containing heptane as solvent was also performed. After 24 hours, the reaction supernatant (after centrifugation to separate the enzyme) was submitted for % m/m and chiral analysis. Conversions of 27% (−)-menthyl acetate (from the available (±) menthol) were obtained for the reactions in MTBE and heptane. An ee of 96.9% and 96.7% was achieved for the reaction in heptane and MTBE respectively.

### EXAMPLE 5

Synthetic (±)-menthol was spiked with increasing levels of the three other pairs of stereoisomers of menthol (produced by the hydrogenation of thymol) and added to Amano AK enzyme in glass reaction vials. The final concentration of other menthol isomers ((±)-isomenthol, (±)-neomenthol and (±)-neoisomenthol) in the reaction was between 0.8 to 3.8% m/v. Vinyl acetate (acyl donor) and heptane (solvent) were added to a final reaction volume of 5 ml. The reaction vials were incubated at 40° C. for 24 hours. After centrifugation to separate the enzyme from the reaction mixture, the supernatant was submitted for % m/m and chiral analysis by GC methods. In the presence of up to 3.8% m/v concentrations of neomenthol, neoisomenthol and isomenthol, ca. 18-20% of the available synthetic (±)-menthol was converted to (−)-menthyl acetate. The enantiomeric excess remained between 96.7 to 96.8%.

### EXAMPLE 6

Reactions with Amano AK enzyme were performed in which the ratio of isomenthol to menthol was increased up to a maximum of 19.6% m/m isomenthol. Vinyl acetate (acyl donor) and heptane (solvent) were added to a final reaction volume of 5 ml. The vials were incubated at 40° C. for 24 hours. After centrifugation to separate the enzyme from the reaction mixture, the supernatant was submitted for % m/m and chiral analysis by GC methods. In the presence of increasing isomenthol concentrations, ca. 18-20% of the available synthetic (±)-menthol was converted to (−)-menthyl acetate. The enantiomeric excess remained between 96.7 to 97.2%.

### EXAMPLE 7

A reaction in which a feed of 50% (±)-menthol and 50% (±)-isomenthol was used was carried out on a 10 mL sale. Amano AK enzyme, vinyl acetate and heptane made up the balance of the reaction mixture. The reaction was carried out at 40° C. for 24 hours. Four cycles were performed by washing the enzyme (after centrifugation) with heptane and adding fresh substrate ((±)-menthol, vinyl acetate and heptane) after every cycle. Conversion of available (±)-menthol was between 12 and 25% over the 4 cycles. Of the isomenthol, 1.5% was converted to isomenthyl acetate. The enantiomeric excess remained 96.4 and 97.2% in the presence of high isomenthol concentrations.

### EXAMPLE 8

A set of experiments was carried out using different ratios of synthetic (+)-menthol and synthetic (−)-menthol, with all other variables (temperature, concentration of (+)-menthol, enzyme moading and amount of vinyl acetate) kept constant. Each of the experiments was carried out at 40° C., with 26% total menthol concentration (% m/m), and with one equivalent of vinyl acetate with respect to total menthols present. Free Amano AK enzyme was used.

Each of the reactions was carried out on a 15 g-20 g scale in Multireactors. A typical experimental procedure was: Heptane (10.52 g), synthetic (−)-menthol (ex Aldrich, 4.49 g) and vinyl acetate (2.65 mL) were transferred to the reactor. Enzyme (first cycle free Amano AK, 337.5 mg) was added when the temperature was at about 25° C., and the reactors were then heated to 40° C. with stirring. Samples were taken after 8 hours and after 24 hours, and analysed by qualitative GC to obtain conversion data, and in cases where (±)-menthol mixtures were used, samples were analysed by chiral GC for enantiomeric excess determination.

The conversion and enantiomeric excess (ee) results are summarised below.

| | | |
|---|---|---|
| Initial % | | |
| (−) | 8 hours | hours |

| menthol | % Conv. | % ee | % Conv. | % ee |
|---|---|---|---|---|
| 75 | 7.54 | 99.35 | 33 | 99.05 |
| 60 | 6.36 | 98.41 | 27.8 | 98.19 |
| 50 | 6.26 | 97.81 | 25.7 | 97.19 |

### EXAMPLE 9

A batch reaction (330 mL) was performed at 35° C. with 42.6% m/v (±)-menthol (2.3M), vinyl acetate (in a 1:1 molar ratio of vinyl acetate to (−)-menthol) and Amano AK enzyme. Heptane was used as a solvent in the reaction. After 23 hours, a sample from the reaction was filtered and submitted for % m/m and chiral analysis by GC methods. The results showed a conversion of 25% and the (−)-menthyl acetate that formed had an ee of 96.3%.

1. A process of separating a desired (−) stereoisomer which is selected from (−) menthol or an equivalent (−) compound where the isopropyl group is replaced with an isopropanol or an isopropylene group, from a starting material comprising:
(a) 40 to 100 m/m % of a mixture of (−)-menthol and (+)-menthol;
(b) up to 30 m/m % of a mixture of (−)-isomenthol and (+)-isomenthol;
(c) up to 20 m/m % of a mixture of (−)-neomenthol and (+)-neomenthol; and
(d) up to 10 m/m % of a mixture of (−)-neoisomenthol and (+)-neoisomenthol,
or an equivalent (±) mixture where the isopropyl group is replaced with an isopropanol or an isopropylene group, including the steps of:
(1) contacting the starting material with an esterifying agent and a stereospecific enzyme which is a Pseudomonas lipase enzyme which stereoselectively esterifies the —OH group of the desired (−) steroisomer, for a time sufficient to convert a desired percentage of the desired (−) stereoisomer to a desired (−) esterified compound where the —OH group is converted to a group —O—C(O)—R₄, wherein R₄ is an alkyl or an aryl group or hydrogen, to give a first reaction product including the desired (−) esterified compound, the organic solvent, the unconverted stereoisomers, excess esterifying agent and by-products of the reaction; and
(2) separating the desired (−) esterified compound from the first reaction product.

2. A process according to claim 1 wherein step (2) comprises the sub-steps of:
(2)(a) separating the first reaction product from the enzyme;
(2)(b) removing the organic solvent, the excess esterifying agent, and the by-products of the reaction to give a second reaction product; and
(2)(c) separating the desired (−) esterified compound from the second reaction product to give a third reaction product containing the unconverted stereoisomers.

3. A process according to claim 1 or claim 2 including the following step prior to step (1) of:
(I) subjecting a racemic mixture of the eight stereoisomers of a compound of the formula III. 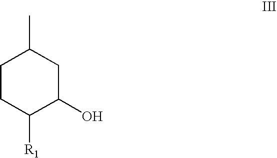
wherein R₁ represents an isopropanol group, an isopropyl group or an isopropylene group, to a distillation step to separate at least a portion of one or more of the (±) mixtures of isomenthol, neomenthol and neoisomenthol or their equivalents where the isopropyl group is replaced with an isopropanol or an isopropylene group, from the (±) mixture of menthol or its equivalent where the isopropyl group is replaced with an isopropanol or an isopropylene group, to give the starting material for step (1).

4. A process according to claim 3 including the step, after step (2) of:
(3) racemizing any unconverted desired (−) stereoisomer, and the other unconverted stereoisomers in the third reaction product, and the six other stereoisomers of the compound of the formula III obtained in the step (I), to give a fourth reaction product containing a mixture approaching the thermodynamic equilibrium of the eight stereoisomers and recycling the fourth reaction product to step (I).

5. A process according to claim 4 including the step, after step (3) of:
(4) hydrolysing the desired (−) esterified compound to give the desired (−) stereoisomer.

6. A process according to claim 5 wherein when the desired (−) stereoisomer or the desired (−) estenfied compound has an isopropanol group or an isopropyl group, before or after step (4), the desired (−) esterified compound or the desired (−) stereoisomer is subjected to a reduction step to convert the isopropanol or the isopropylene group to an isopropyl group.

7. A process according to any one of claims 1 to 6 wherein in step (1) the starting material comprises:
(a) about 80 m/m % of a mixture of (−)-menthol and (+)-menthol,
(b) about 10 m/m % of a mixture of (−)-isomenthol and (+)-isomenthol;
(c) about 6 m/m % of a mixture of (−)-neomenthol and (+)-neomenthol; and
(d) about 4 m/m % of a mixture of (−)-neoisomenthol and (+)-neoisomenthol;
or an equivalent (±) mixture where the isopropyl group is replaced with an isopropanol or an isopropylene group.

8. A process according to any one of claims 1 to 7 wherein in step (1) the enzyme is Amano AK lipase enzyme.

9. A process according to any one of claims 1 to 8 wherein step (1) is carried out using a suitable organic solvent.

10. A process according to claim 9 wherein in step (1) the solvent is selected from the group consisting of t-butyl methyl ether; cyclohexane; hexane; heptane; and isooctane.

11. A process according to claim 10 wherein in step (1) the solvent is n-heptane.

12. A process according to any one of claims 1 to 11 wherein in step (1) the esterifying agent is selected from the group consisting of vinyl acetate, butyl acetate, octanoic acid, isopropenyl acetate, vinyl butyrate, ethyl lactate and ethyl acetate.

13. A process according to claim 12 wherein in step (1) the esterifying agent is vinyl acetate.
